(19) United States
(12) Patent Application Publication
(10) Pub. No.: US 2010/0256468 A1
(43) Pub. Date: Oct. 7, 2010

(54) APPARATUS FOR MEASURING BIOLOGICAL LIGHT

(75) Inventors: Naoki Tanaka, Tokyo (JP); Masashi Kiguchi, Kawagoe (JP); Atsushi Maki, Fuchu (JP); Shingo Kawasaki, Kashiwa (JP); Noriyoshi Ichikawa, Moriya (JP); Fumio Kawaguchi, Hinode (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(73) Assignee: HITACHI MEDICAL CORPORATION, Tokyo (JP)

(21) Appl. No.: 12/600,285

(22) PCT Filed: Feb. 1, 2008
(86) PCT No.: PCT/JP2008/051628

§ 371 Date: May 13, 2010

(30) Foreign Application Priority Data
May 21, 2007 (JP) 2007-133766

(51) Int. Cl. A61B 5/1455; G06N 5/02

(52) U.S. Cl. 600/323; 706/54

(57) ABSTRACT

The efficiency of separating normal control subjects from non-normal control subjects and separating disorders from one another is improved. Plural classification models having a stratified structure are used, and areas from which measurement data used in the plural classification models are acquired differ among the classification models.

### TECHNICAL FIELD

The present invention relates to an apparatus for measuring biological light which non-invasively supports diagnosis of disorder.

### BACKGROUND ART

An apparatus for measuring biological light is capable of non-invasively measuring local changes in hemoglobin in a living organism. This is a method of measuring a changing amount of hemoglobin by irradiating a test subject with light beams having wavelengths from the visible range to the infrared range, and by detecting, with a single photodetector, the light beams of plural signals having passed through the inside of the test subject. This method is characterized by restraining less a test subject than such cerebral function measuring techniques as MRI and PET.

As for one of clinical applications of this apparatus, it has been reported that the change pattern in hemoglobin in the frontal lobe of a patient of such psychiatric disorders as depression and schizophrenia has a specific characteristic that is not observed in those of healthy normal subjects (Non-Patent Documents 1 and 2). Specifically, there have been found characteristics in which an integral (area) of the temporal wave of hemoglobin of each of test subjects performing a given task is large for healthy normal subjects, small for depression patients, and moderate for schizophrenia patients. In addition, it is observed that the hemoglobin in the schizophrenia patients increases again to form a second peak after task. According to a method disclosed in WO 2005/025421 A1, characteristic parameters are firstly extracted from the wave of a measured hemoglobin change. Then, the Mahalanobis distances between these characteristic parameters and data in a database of disorders are calculated to obtain disorder deciding scores. The scores thus obtained are displayed. In this method, the database of disorders used as the reference for decision are classified and categorized in accordance with the names of disorders given by the user.

[Patent Document 1] WO 2005/025421 A1

[Patent Document 2] WO 2006/132313 A1

[Non-Patent Document 1] Fukuda Masato et al., “Near-infrared spectroscopy as a laboratory test for diagnosis and treatment of psychiatric disorders in clinical practice” Brain Science and Mental Disorders, vol. 14, no. 2 (2003), pp. 155-71.

[Non-Patent Document 2] Fukuda Masato, “Dynamics of Local Cerebral Blood Flow in the Frontal Lobe in Psychoneurotic Disorders—Study Using Optical Topography” Japan Society for the Promotion of Science Grants-in-Aid, Report of Research Results Fiscal Years 2001 to 2002 (Heisei 13 to 14).

### DISCLOSURE OF THE INVENTION

### Problems to be Solved by the Invention

The above-described techniques, however, has an aspect of failing to separate the normal controls from the non-normal controls with sufficient accuracy. Moreover, in some cases, categories for separating disorders from each other are not formed based on characteristics of wave in a simple manner.

### Means for Solving the Problems

The present invention focuses on measured area dependency of the temporal wave. Without restricting the measurement area to the frontal lobe, the present invention measures the hemoglobin in plural areas, such as the frontal lobe, the right and left temporal lobes, and the parietal lobe. Then, disorders are classified by using, as characteristic parameters, the slope immediately after task start, the integral (area) during task, the second peak area after task, the center of balance for the entire wave, and the like, which are obtained from the measured temporal waves of hemoglobin. In this way, the normal controls can be separated from the non-normal controls more effectively. In addition, employing stratified classification of disorders enables category formation in a simper manner.

### EFFECTS OF THE INVENTION

The present invention makes it possible to non-invasively provide information to support diagnosis of disorders.

### BRIEF DESCRIPTION OF THE DRAWINGS

FIG. 1 is a diagram illustrating the configuration of an apparatus.

FIG. 2 shows charts illustrating characteristics of waves for disorders.

FIG. 3 is a chart illustrating a method of extracting characteristic parameters from a wave of a hemoglobin change.

FIG. 4 is a diagram illustrating the concept of a stratified classification.

FIG. 5 is a chart illustrating a classification model for classification into a Type 1 group and non-Type 1 group.

FIG. 6 is a chart illustrating a classification model used to classify the non-Type 1 group into three different groups.

FIG. 7 is a scatter diagram of measured data.

FIG. 8 is a chart illustrating a classification model for classification into a Type 2 group and a Type 3 group.

FIG. 9 is a flowchart illustrating a disorder-state decision operation.

FIG. 10 shows charts illustrating tendencies in the scatter diagram.

FIG. 11 is a diagram illustrating an exemplar disorder-decision algorithm.

FIG. 12 is a diagram illustrating in detail the configuration of the apparatus.

FIG. 13 is a diagram illustrating an input screen.

FIG. 14 is a diagram illustrating an input screen used to specify a measured area for the data used to classification.

### DESCRIPTION OF SYMBOLS

10: part for biometry
20: part for calculating characteristics
30: part for input
40: part for decision
50: part for recording
60: display
101: oscillator
102: light source
103: optical fiber
104: coupler
105: optical fiber
106: measurement target (test subject)
107: optical fiber for light reception
108: photoreceiver
109: lock-in amplifier
110: analog-to-digital converter
111: calculator for controlling measurement
112: calculator

### BEST MODES FOR CARRYING OUT THE INVENTION

For an embodiment, disorder decision using an apparatus according to the present invention is performed on a total of 107 test subjects of the following four groups: a normal control group; a schizophrenia patient group; a depression patient group; and a bipolar disorder patient group.

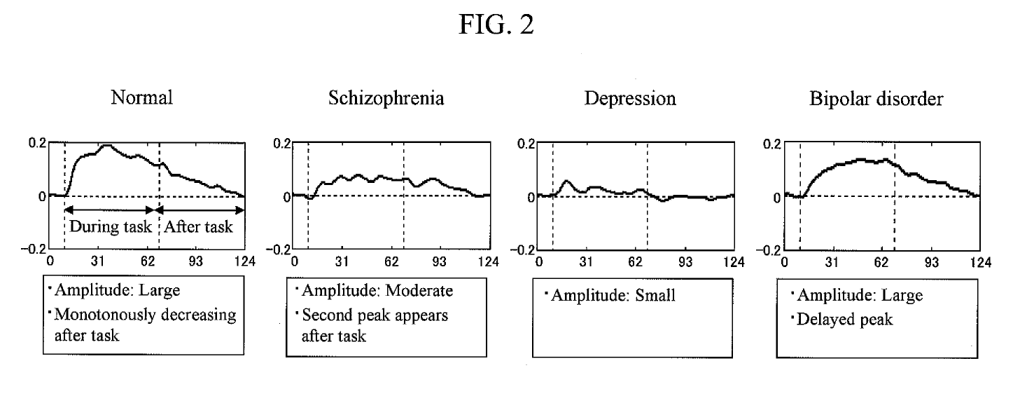

FIG. 2 shows charts illustrating characteristic wave patterns of disorders, which are reported in Fukuda et al. (see Non-Patent Document 1 and 2). A verbal fluency task including word recall is given to the test subjects. The characteristic wave pattern for the normal control group includes large changing in hemoglobin and a monotonous decrease in hemoglobin after task. The characteristic wave pattern for the schizophrenia patient group includes moderate changing in hemoglobin and a second peak in the wave after task. The characteristic wave pattern for the depression patient group includes small changing in hemoglobin. The characteristic wave pattern for the bipolar disorder patient group includes large changing in hemoglobin and a peak appearing in the latter half of the period during which the test subjects perform the task.

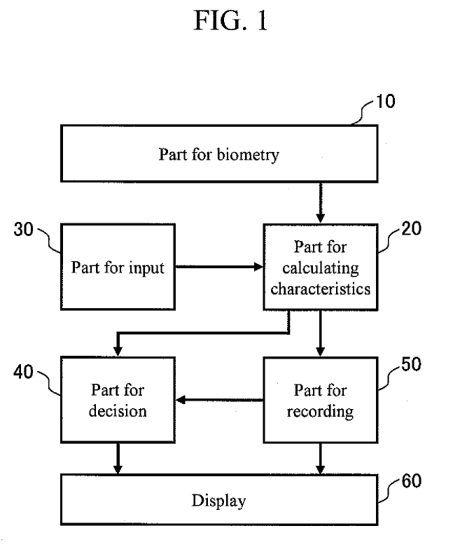

The apparatus for supporting diagnosis of disorders according to the present invention quantifies the above-mentioned characteristics, and automatically classifies the waves on the basis of the quantified characteristics. FIG. 1 shows an exemplar configuration of the apparatus. The apparatus for supporting diagnosis of disorders includes: a part for biometry 10 that measures the change in hemoglobin caused when a verbal fluency task including word recall is given to each test subject, the measurement being made by, for example, the operator's input; and a part for calculating characteristics 20 that calculates the characteristic parameters of the measured wave. The characteristic parameters thus calculated are stored, together with the results of the definitive diagnosis, in a part for recording 50. In addition, the characteristic parameters are sent to a part for decision 40, where a decision is made using the characteristic parameters. As a result of the decision, each of the test subjects is classified into any of five types. The classification results, together with the information enrolled in the database are displayed on a display 60.

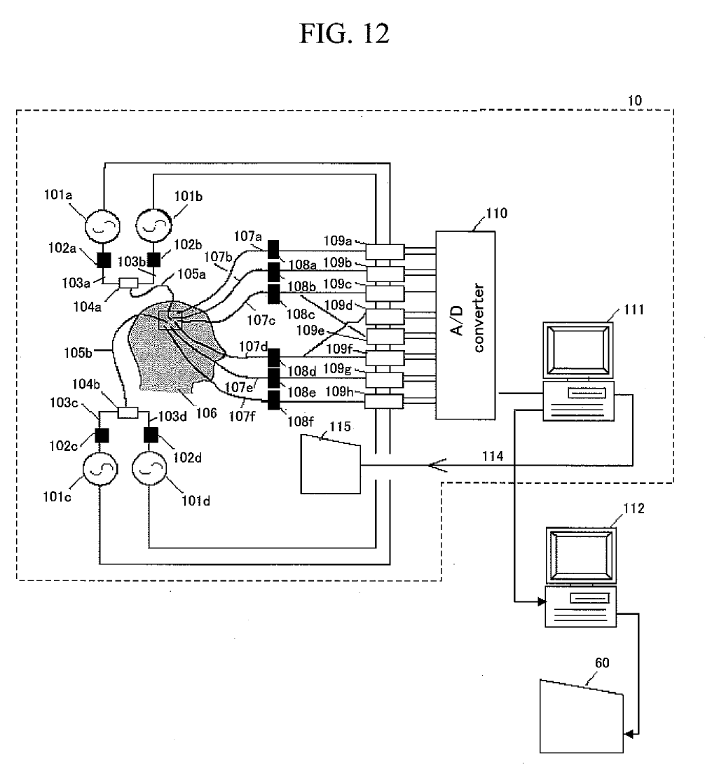

Subsequently, the configuration of the apparatus will be described in more detail by referring to FIG. 12. To obtain the local changing amount of oxyhemoglobin, the local changing amount of deoxyhemoglobin, and the total changing amount of hemoglobin, the part for biometry 10 is used to irradiate plural areas of the head of each test subject with light beams having wavelengths from the visible range to the infrared range. Then, the light beams of the plural signals that have passed through the inside of the test subject are detected and measured by a single photodetector.

The apparatus for supporting diagnosis of disorders according to the present invention includes: plural light sources 102a to 102d; a modulator; plural means for light emission; and plural means for light reception. The plural light sources 102a to 102d emit light beams having different wavelengths (780-nm wavelength for light sources 102a and 102c and 830-nm wavelength for light sources 102b and 102d). The modulator includes oscillators 101a to 101d (101c and 101d) to respectively modulate the intensities of the light beams emitted from the plural light sources 102a and 102b (102c and 102d), the oscillators 101a to 101d (101c and 101d) having different frequencies from each other. The means for light emission include couplers 104a and 104b each of which couples together the light beams whose intensities have been modulated, via optical fibers 103a and 103b (103c and 103d), respectively, and emits the coupled light beams through optical fibers for light emission 105a (105b). The means for light emission irradiates different positions of the scalp of the object to be tested, that is, of a test subject 106, with the light beams from the couplers 104a and 104b, respectively. The means for light reception respectively include: plural optical fibers for light reception 107a to 107d; and photoreceivers 108a to 108f provided respectively in the optical fibers for light reception 107a to 107d. An end of each of the optical fibers for light reception 107a to 107d is located in the vicinity of each of the positions irradiated by the plural means for light emission. The distance between each light irradiation position and the end of each of the optical fibers for light reception 107a to 107d is kept constant (e.g., 30 mm in this case). The six optical fibers for light reception 107a to 107f collect the light that has passed through the living organism, and the light having passed through the living organism and then collected by the optical fibers for light reception 107a to 107f are subjected to photoelectric conversion respectively by the photoreceivers 108a to 108f. The means for light reception detect the light reflected inside of the test subject, and convert the detected light to electric signals. Photoelectric conversion elements are used as the photoreceivers 108. Photomultiplier tubes and photodiodes are some examples of such photoelectric conversion elements.

The electric signals that represent the intensities of the light having passed through the living organism and are subjected to the photoelectric conversion by the photoreceivers 108a to 108f (hereinafter, the electric signal will be referred to as “living-organism-passed-light intensity signal”) are inputted into lock-in amplifiers 109a to 109h. Note that the photoreceivers 108c and 108d detect the intensities of the light having passed through the living organism and been collected respectively by the optical fibers for light reception 107c and 107d, each which is positioned equidistantly from both of the optical fibers for light emission 105a and 105b. Accordingly, the signal detected by the photoreceiver 108c (108d) is divided into two different lines, and the signals of the two lines are inputted respectively into the lock-in amplifiers 109c and 109e (109d and 1090. The intensity modulating frequencies of the oscillators 101a and 101b are inputted as the reference frequencies into the lock-in amplifiers 109a to 109d whereas the intensity modulating frequencies of the oscillators 101c and 101d are inputted as the reference frequencies into the lock-in amplifiers 109e to 109h. Consequently, the lock-in amplifiers 109a to 109d output, separately, the living-organism-passed-light intensity signals corresponding to the light sources 102a and 102b whereas the lock-in amplifiers 109e to 109h outputs, separately, the living-organism-passed-light intensity signals corresponding to the light sources 102c and 102d.

The passed-light intensity signals of various wavelengths separately outputted by the lock-in amplifiers 109e to 109h are subjected to analog-to-digital conversion by an analog-to-digital converter (AJD converter) 110. Then, the resultant signals are sent to a calculator for controlling measurement 111. The calculator for controlling measurement 111 uses the passed-light intensity signals to calculate, from the detection signals at the detection points, the relative changing amount of the oxyhemoglobin concentration, that of the deoxyhemoglobin concentration, and that of the total hemoglobin concentration. The calculation is performed in accordance with relies on the method described in Non-Patent Document 1. The relative changing amounts thus obtained are stored, in a storage, as time-series data for the plural measurement points. Although FIG. 12 shows only one measurement point, the measurement is actually performed simultaneously at plural areas such as the frontal lobe, the right and left temporal lobes, and the parietal lobe. The hemoglobin wave, which will be described later, is acquired for each of the areas.

The foregoing description is based on an embodiment where plural kinds of light are separated by a modulation method, but this is not the only possible form. For example, a time-division method may be employed, instead. Specifically, plural kinds of light are discriminated from one another by emitting the plural light at different timings.

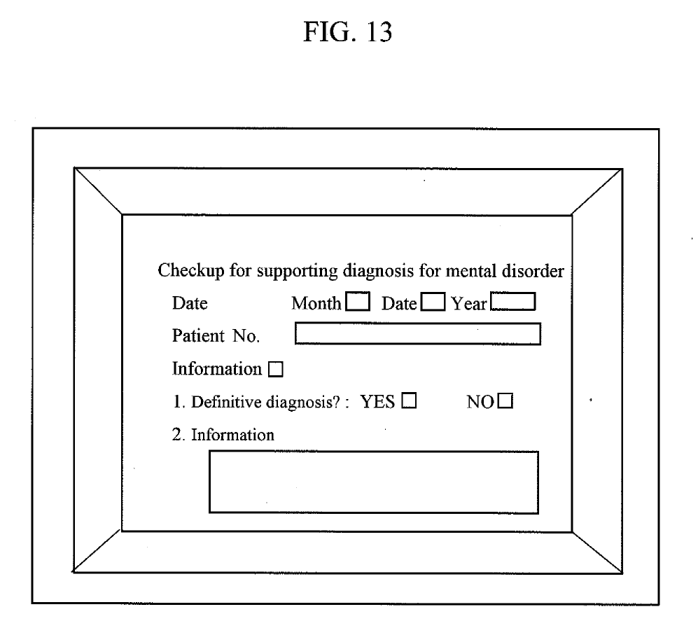

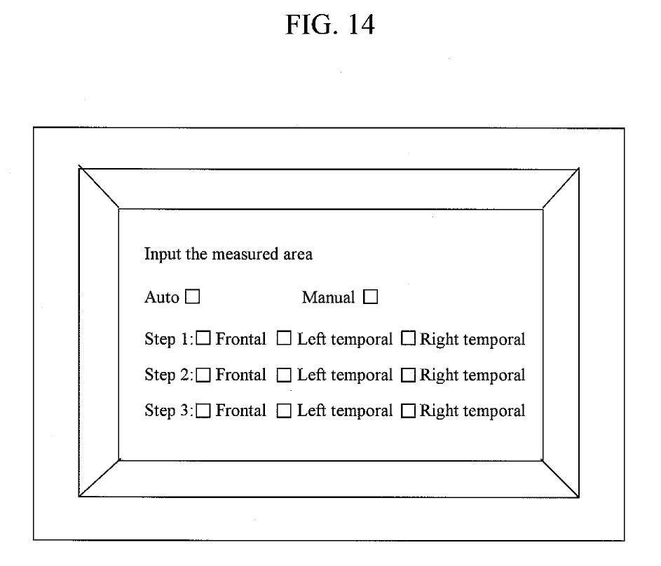

A part for input 30, the part for calculating characteristics 20, the part for recording 50, the part for decision 40 are all located in a calculator 112. The part for input 30 is used to input information that is necessary to decide the disorder. The information is inputted using an input screen as shown in FIG. 13. Each test subject is identified using a test-subject number, but his/her name may be used instead. If there is a definitive-diagnosis result, the checkbox of YES for item 1 has to be checked. In this case, the test result is automatically stored in the database. The next input screen (shown in FIG. 14) is used for specifying the measured area of data to be used in the classification. If some predetermined measured-area data are used, the checkbox of Auto has to be checked. If the operator him/herself specifies the measured areas, the checkbox of Manual has to be checked. If Manual is selected, the operator has to check the checkboxes to specify which of the measured-area data is used in each of the first to third stages.

The part for calculating characteristics analyzes the characteristic parameters on the basis of the wave data of the measured local changing amount of oxyhemoglobin, that of deoxyhemoglobin, and the measured total changing amount of hemoglobin. The wave data and the characteristic parameters, together with the information on the measured areas, are sent to the part for recording located in the calculator 112. The part for recording temporarily stocks the measurement information on the test subjects so as to make the execution of the subsequent processing possible. In addition, if there is a definitive diagnosis for a test subject, the part for recording may also function as a database to store the measurement information. The information stocked in the database may be used for automatically adjusting the parameters, which will be described later. In addition, the information stocked in the database may also be used for diagnosing a patient by use of this apparatus. The part for decision located in the calculator 112 makes the decision of disorders by a method that will be described later. The display 60 displays the result of the decision.

Note that the calculator 111 and the calculator 112 are depicted as different calculators in FIG. 12, but it is, naturally, possible to use only a single calculator instead.

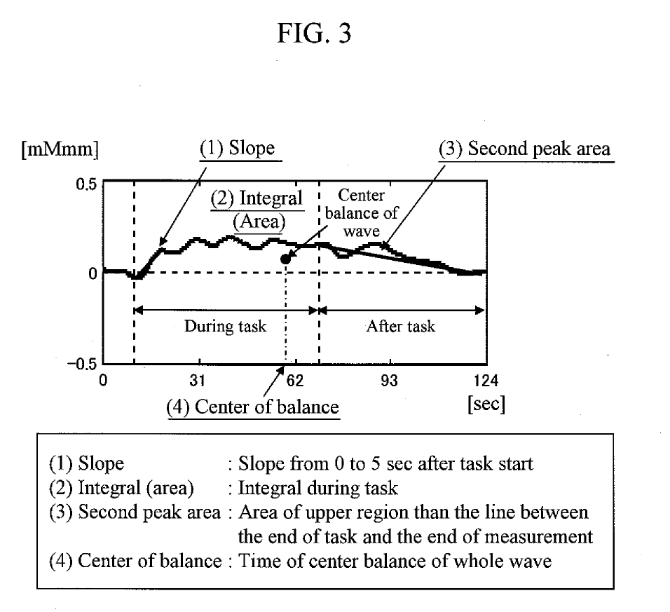

FIG. 3 is a chart illustrating a way of calculating various kinds of characteristic parameters ((1) slope; (2) integral (area), (3) second peak area, and (4) center of balance) from a measured hemoglobin wave. The slope is a parameter representing the response speed to the task, and is calculated from the slope of a section of the hemoglobin wave from 0 to 5 seconds after task start. The integral (area) is considered as a parameter representing the magnitude of the response, and is calculated by integrating the hemoglobin wave of a section corresponding to the period during task. The second peak area is considered as a parameter representing a psychological tendency to disobey the command to finish the task. The second peak area is calculated as an area above the line connecting the value of the hemoglobin at the end of task to the value of the hemoglobin at the end of measurement. The center of balance is considered as a parameter representing the durable response speed, and is defined as the relative time at which the center of balance for the wave is positioned. Note that the relative time is defined by giving a value 0 to the time of starting measurement and a value 1 to the time of finishing measurement. These characteristic parameters are acquired for each of the hemoglobin waves obtained by the plural measurement channels. Either the maximum value or the average value obtained through the channels for the frontal lobe, and either the maximum values or the average values obtained through the channels for the right and left temporal lobes are used as the representative values for each test subject. The representative values obtained as the maximum values or the average values may be ones for the entire frontal lobe, for the entire right temporal lobe, and for the entire left temporal lobe. Alternatively, the representative values obtained as the maximum values or the average values may be ones obtained through either a single specific one or plural specific ones of the channels for the frontal lobe, for the right temporal lobe, and for the left temporal lobe.

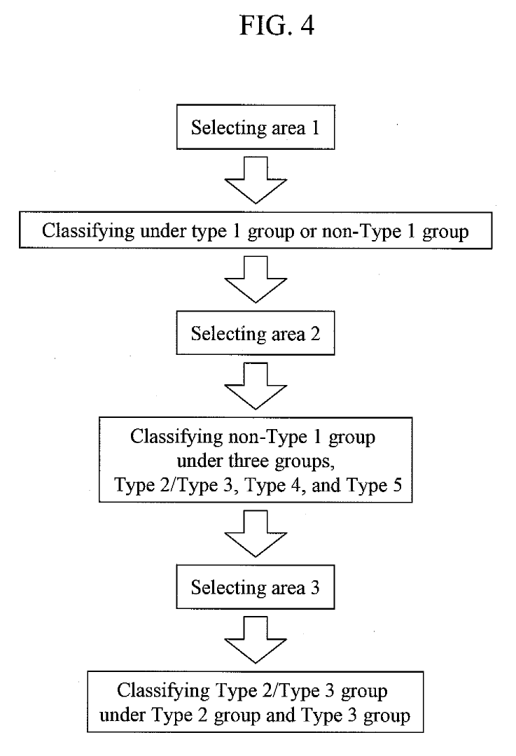

FIG. 4 is a diagram illustrating the classification method of this embodiment using the data measured in the plural areas. The apparatus of the present invention classifies the measured waves into five different types. Incidentally, each of the healthy subjects and of the disorder patients reacts the task in a very complex way. The complexity makes it difficult to classify the waves at a single stage using data measured in a single area even if the above-mentioned four different kinds of characteristic parameters are combined together. In the verbal fluency task employed in this embodiment, the test subjects were instructed to give as many words as possible each of which starts one of the given sounds “a,” “ka,” and “sa,” and to repeat pronouncing “a-i-u-e-o” in a uniform rhythm during rest periods. Various functions that locally exist in different areas of the cerebral cortex are presumably relevant to the performing of the task. Some examples of such functions are: the auditory function; the short-term memory function; the language function; the search function from long-term memory; and the motor function.

Although not yet become academically-established, there is a reported fact that language dysfunction (locally existing in the left temporal lobe in most cases) and a functional decline of the frontal lobe are observed in schizophrenia patients and that a functional decline of the frontal lobe and the like are observed in depression patients. It is possible that different disorders cause the functional declines and/or the dysfunction of different areas and of different degrees to take place. This is why the present invention employs the classification based on the data measured in plural areas. The test subjects herein are first classified into two groups: a Type 1 group and a non-Type 1 group. Then, those in the non-Type 1 group are classified into three groups: a Type 2/Type 3 group; a Type 4 group; and a Type 5 group. Finally, those in the Type 2/Type 3 group are classified into two groups: a Type 2 group and a Type 3 group. Note that, as will be shown below, the Type 1 group includes mainly the normal control subjects (hereinafter, sometimes abbreviated as “NC”). Each of the Type 2 group and the Type 5 group includes mainly the schizophrenia patients (hereinafter, sometimes abbreviated as “SC”). The Type 3 group includes the bipolar disorder patients (hereinafter, sometimes abbreviated as “BP”). The Type 4 group includes mainly the depression patients (hereinafter, sometimes abbreviated as “DP”). The use of the data for the appropriate measured areas for each stage characterizes the present invention.

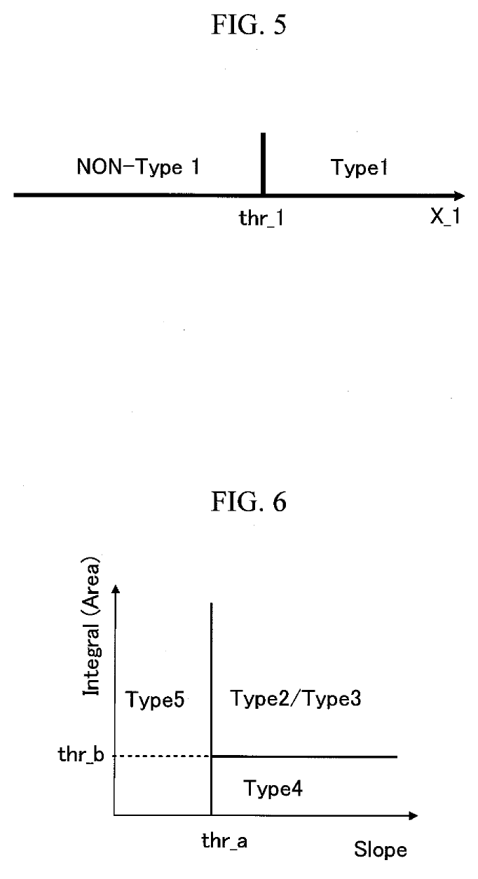

FIG. 5 illustrates one of the models for the above-described classifications that is employed for classifying the test subjects into the Type 1 group and the non-Type 1 group. A variable X_1 is defined by the following equation (1) formulated by normalizing the above-mentioned four different kinds of characteristic parameters and connecting and synthesizing the normalized parameters into a linear form. If the value of the variable X_1 is equal to or larger than thr_1, the test subject of that wave belongs to the Type 1 group. The underlined parameters in the equation (1) are the normalized parameters. Note that the “normalization” used here is a linear transformation by which each variable has an average of zero and a variance of one.

X_{—}1=C1*integral (area)+C2*slope+C3*second peak area+C4*center of balance (1)

Using the data measured at the frontal lobe and assuming that C1=0.33, C2=0.13, C3=−0.62, C4=−0.70, and thr_1=0.482, it was decided that 36 of all the 107 cases belonged to the Type 1 group. While 67% of the normal control subjects were decided to belong to the Type 1 group, only 6% of the non-normal control subjects were decided to belong to the Type 1 group. When C4 was fixed at zero, the assumption that C1=0.56, C2=0.42, C3=−0.71, and thr_1=0.191 resulted in the highest coincidence ratio with the diagnosis labels. In this case, it was decided that 46% of the normal control subjects and 31% of the non-normal control subjects belonged to the Type 1 group. When, in addition, C3 was fixed at zero, it was decided that 21% of the normal control subjects and the 48% of the normal control subjects belonged to the Type 1 group. When the data measured in the right and left temporal lobes, it was decided that no more than 32 of all the 107 cases belonged to the Type 1 group irrespective of the values of C1, C2, C3, C4, and thr_1. It was decided that only 49% of the normal control subjects, at most, belonged to the Type 1 group. In this case, it was decided that 19% of the non-normal control subjects belonged to the Type 1 group. These facts reveal that the use of the data measured in the frontal lobe is important for separating the Type 1 group from the non-Type 1 group and that the center of balance and the second peak area are the important characteristic parameters.

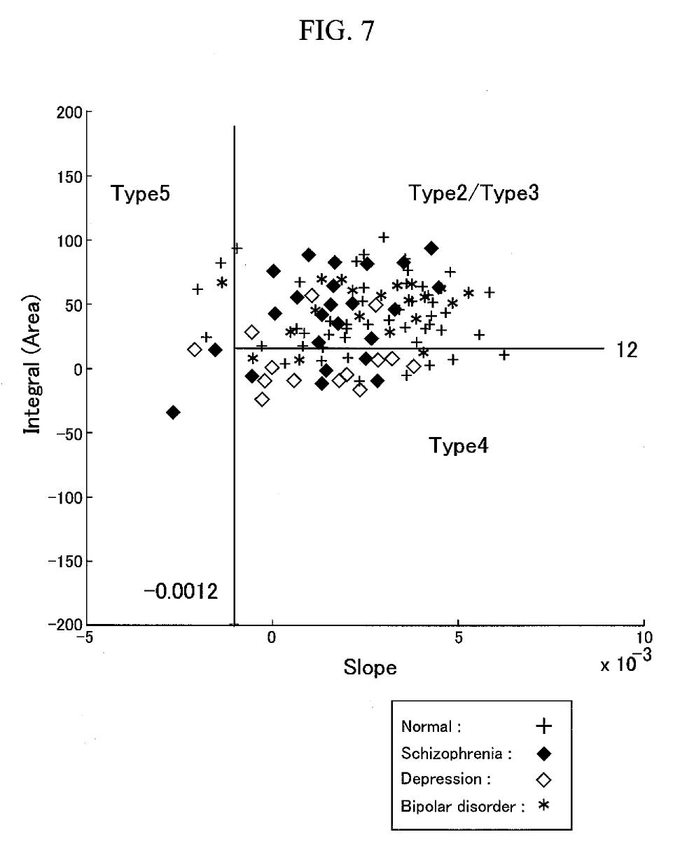

FIG. 6 illustrates one of the above-described classification models that is employed for classifying the test subjects belonging to the non-Type 1 group into the Type 2/Type 3 group, the Type 4 group, and the Type 5 group. If the slope is smaller than thr_a, it is decided that the subject belongs to the Type 5 group. If the slope is equal to or larger than thr_a, and, at the same time, if the integral (area) is equal to or larger than thr_b, it is decided that the subject belongs to the Type 2/Type 3 group. If the slope is equal to or larger than thr_a, and, at the same time, if the integral (area) is smaller than thr_b, it is decided that the subject belongs to the Type 4 group. In this embodiment, the use of the data measured in the left temporal lobe combined with an assumption that thr_a=−0.0012, and thr_b=12 resulted most favorably. FIG. 7 shows a scatter diagram for this case. FIG. 7 shows that the four groups are not clearly separated from one another, but rather distributed as each group lies over the others to a significant degree. A small part of the schizophrenia patients belongs to a region with a small slope, that is, to the Type 5 group. The rest of the schizophrenia patients, together with most of the bipolar disorder patients, are distributed in a region with both a large slope and a large integral (area), that is, to the Type 2/Type 3 group. Most of the depression patients are distributed in a region with a relatively large slope and a small integral (area), that is, to the Type 4 group. Since there are a large total number of normal control subjects, quite a number of normal control subjects are distributed in the region corresponding to the non-Type 1 group, especially in the region corresponding to the Type 2/Type 3 group.

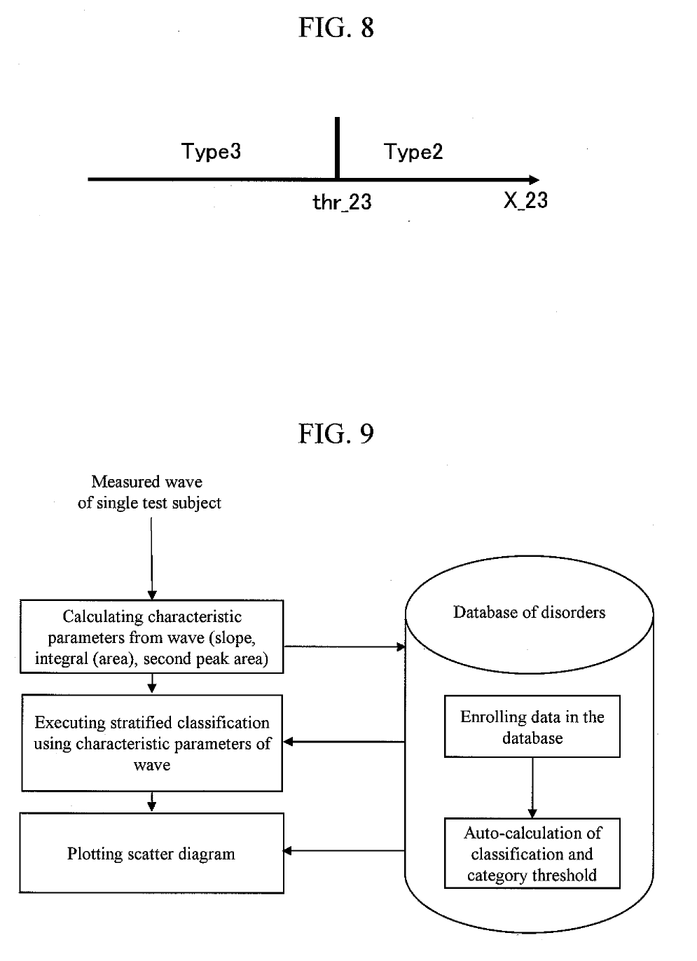

FIG. 8 illustrates one of the above-described classification models that is employed for classifying the test subjects belonging to the Type 2/Type 3 group into the Type 2 group and the Type 3 group. A variable X_23 is defined by the following equation (2) formulated by normalizing the above-mentioned four different kinds of characteristic parameters and connecting and synthesizing the normalized parameters into a linear form. If the value of the variable X_23 is equal to or larger than thr_23, the test subject of that wave is decided to belong to the Type 2 group. Otherwise, the test subject is decided to belong to the Type 3 group. The underlined parameters in the equation (2) are the normalized parameters.

X_{—}23=D1*integral (area)+D2*slope+D3*second peak area+D4*center of balance (2)

Using the data measured at the frontal lobe and assuming that D1=0.15, D2=0.15, D3=0.98, D4=0.0, thr_1=0.129, it was decided that 18 of all the 38 cases belonged to the Type 2 group. When each Type group was identified with the label of its main component disorder, the final coincidence ratios of the subjects of each disorder with the diagnosis label were: 67% for NC; 70% for C; 68% for DP; and 66% for BP. When D3 was fixed at zero, the assumption that D1=0.55, D2=0.76, D3=0.0, D4=0.34, and thr_1=0.349 resulted in the highest average coincidence ratio with the diagnosis labels, but the highest average coincidence ratio was no higher than 39%. In addition, the second peak area is the important characteristic parameter for separating the Type 2 group from the Type 3 group.

As has been described thus far, in this embodiment, the highest coincidence ratio with the diagnosis label was obtained when the data on the frontal lobe, the data on the left temporal lobe and the data on the frontal lobe were used at the first, second, and third stages, respectively. When other combinations of measured areas were used, the best coincidence ratio resulted from a case where: the data on the frontal lobe, the data on the left temporal lobe, and the data on the right temporal lobe were used at the first, second, and third stages, respectively. Nonetheless, in this case, the coincidence ratios with the diagnosis labels were: 67% for NC; 65% for SC; 55% for DP; and 67% for BP. The average coincidence ratio of this case was not as high as the average coincidence ratio of the case where: the data on the frontal lobe, the data on the left temporal lobe, and the data on the frontal lobe were used at the first, second, and third stages, respectively.

FIG. 9 is a flowchart illustrating an operation in which the characteristic parameters are extracted from the measured wave of a single test subject, and then displayed in a scatter diagram together with the data obtained from the database. The characteristic parameters of the single test subject are compared with the data on the characteristic parameters obtained from the database, and the comparison is displayed in the scatter diagram. Accordingly, the disorder category can be decided while the image of the overall tendency can be caught. The apparatus for measuring biological light can obtain the hemoglobin waves and the characteristic parameters for plural channels at a single measurement. To obtain the characteristic parameters, the average wave of the waves for measured areas was used. Note that, for the second peak area, the largest one of the second peak areas for all the channels was used.

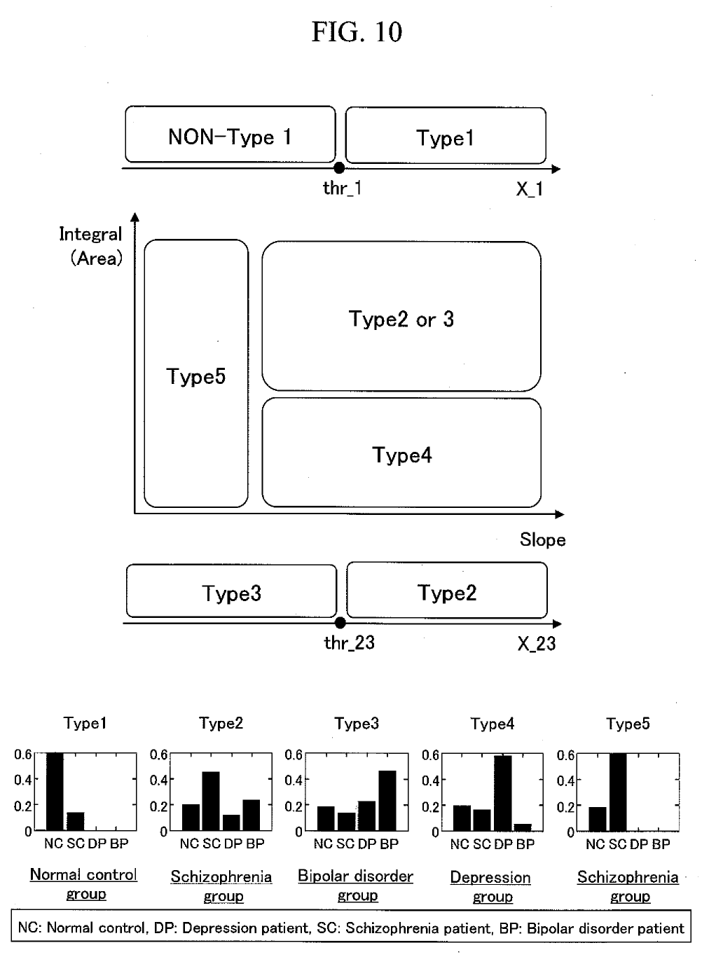

FIG. 10 illustrates how many test subjects of each disorder exist in each of the disorder categories that have been classified into by use of the thresholds. Each bar graph shows the existence ratios of the disorders. FIG. 10 shows that: large numbers of normal control subjects, schizophrenia patients, bipolar disorder patients, depression patients, and schizophrenia patients exist in the Type 1 group, the Type 2 group, the Type 3 group, the Type 4 group, and the Type 5 group, respectively. In the Type 2 group and the Type 3 group, it is observed that the schizophrenia patients tend to be separated from the bipolar disorder patients.

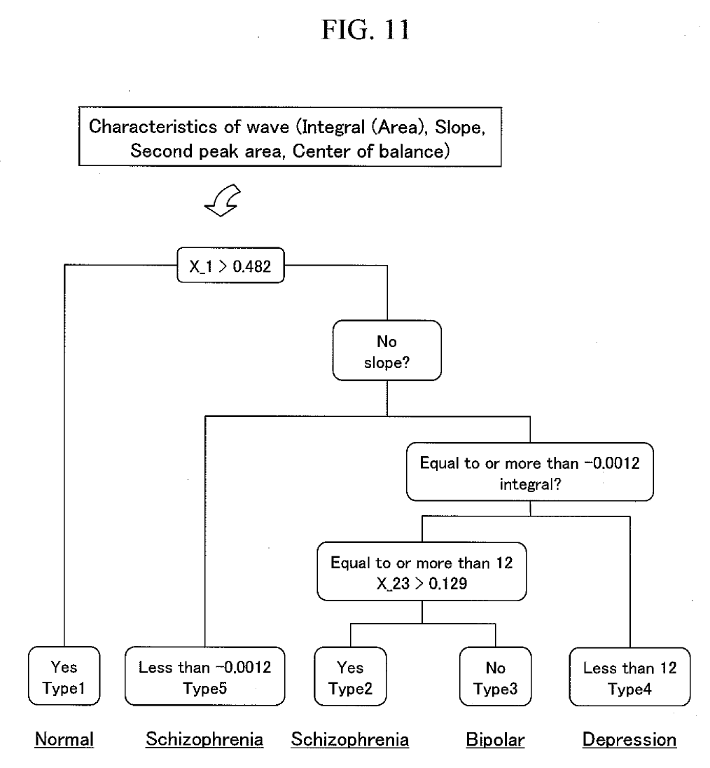

FIG. 11 is a diagram illustrating an exemplar disorder-decision algorithm according to the present invention. By inputting the normalized characteristic parameters calculated from the hemoglobin wave ((1) slope, (2) integral (area), (3) second peak area, and (4) center of balance), the disorder decision can be made. For example, a test subject showing a hemoglobin change with an integral (Area) of 0.65, a slope of 0.71, a second peak area of 0.32, and a center of balance of 0.33 belongs to the normal control (Type 1) group.

The apparatus of the present invention has a function of accumulating data in the database. The data in the database may change, and thus the apparatus has a system for automatically adjusting the classification model along with the change. Description of this system will be given next. Note that automatic adjustment refers to a function of optimizing the parameters used in each model with respect to the data in the database.

Firstly, the optimization of the model described in FIG. 5 will be described. Suppose a four-dimensional space formed by the normalized characteristic parameters. Then, suppose that x represents the inner product of a unit vector c that is directed to a certain direction and a vector x0 corresponding to a piece of data. Subsequently, the minimum value and the maximum value of x are determined using all the data in the database, and then a classification is executed with an appropriate value thr. In this case, p1 is supposed to represent the probability of classifying a normal control subject into the Type 1 group whereas p_1 is supposed to represent the probability of not classifying a non-normal control subject into the Type 1 group. Then, the vector c and the value thr that result in the maximum value for an evaluation function f(u)=u*p1+(1−u)*p_1 are obtained. Note that u=⅓ in this embodiment. The model can be adjusted by using the optimized elements of c (c1, 2, 3, and 4) and thr, and substituting so that C1=c1, C2=c2, C3=c3, C4=c4, and thr_1=thr.

Subsequently, the optimization of the model described in FIG. 6 will be described. This automatic clustering will be described. Assume that j represents a combination of thresholds and the combination j corresponds to three different kinds of Type groups (i.e., TYPE(j,n) n=1, 2, and 3; which corresponds to the above-described Type2/Type3 group, the Type 4 group, and Type 5 group, respectively). The existing probability, in each Type group, for the subjects of each of the disorders such as the normal control subjects, the schizophrenia patients, the depression patients, and the bipolar disorder patients are determined. Note that the probabilities for the subjects of disorders are represented by pNC(j,n), pS(j,n), pD(j,n), and pBP(j,n). A relationship expressed by the following equation exists among these probabilities.

pNC(j,n)+pS(j,n)+pD(j,n)+pBP(j,n)=1

The optimization is executed by selecting the thresholds that make the existing probabilities of the disorders as disproportionate as possible in each of the Type groups. The entropy sum E(j) corresponding to the combination j of thresholds (thr_a, thr_b) can be expressed by the following equations.

$$[ Numerical Expressions 1 ]$$

$$E ( j ) = {\sum }_{n} {p}_{n} E ( j , n )$$

$$E ( j , n ) = - {\sum }_{α = NC , S , D , BP} p α ( j , n ) {log}_{2} p α ( j , n )$$

In the above equations, pₙ represents the proportion of the data included in the Type n when the combination j of thresholds is employed. Here, the combination of the thresholds that results in the minimum entropy sum E(j) is assumed to give the best classification (i.e., the best clustering). Minimizing the entropy corresponds to the threshold selection that makes the existing probabilities of the disorders as disproportionate as possible in each of the Type groups.

Lastly, the optimization of the model described in FIG. 8 will be described. Suppose a four-dimensional space formed by the normalized characteristic parameters. Then, suppose that x represents the inner product of a unit vector c that is directed to a certain direction and a vector x0 corresponding to a piece of data. Subsequently, the minimum value and the maximum value of x are determined using all the data in the database, and then a classification is executed with an appropriate value thr. In this case, p2 is supposed to represent the probability of classifying a schizophrenia patient belonging to the Type 2/Type 3 group into the Type 2 group whereas p_2 is supposed to represent the probability of classifying a bipolar disorder patient belonging to the Type 2/Type 3 group into the Type 3 group. Then, the vector d and the value thr that result in the maximum value for an evaluation function g(w)=w*p2+(1−w)*p2 are obtained. Note that w=½ in this embodiment. The model can be adjusted by using the optimized elements of d (d1, d2, d3, and d4) and thr, and substituting so that D1=d1, D2=d2, D3=d3, D4=d4, and thr_23=thr.

Subsequently, another embodiment that is different from the above-described embodiment will be described. In the apparatus configuration shown in FIG. 12, in this embodiment, the wavelength of each of the light sources 102a and 102c is 690 nm whereas that of each of the light sources 102b and 102d is 830 nm. In the verbal fluency task, the test subjects were instructed to give as many words as possible each of which belongs to a given category, and to repeat pronouncing “a-i-u-e-o” in a uniform rhythm during the rest periods. The categories given as the task were names of animals, names of domestic cities, and names of plants. 20 seconds were given to present the task and to give answers for each category. Accordingly, a total of 60 seconds were secured for performing the task. Good attention has to be paid when the task is selected. It is important that the task to be selected should be easy. The verbal fluency task of this embodiment differs from the verbal fluency task of the previous embodiment in the following point. The condition for giving words in the previous embodiment is that the initial sound of each word has to coincide with the given sound whereas the condition in this embodiment is that the category to which each word belongs has to coincide with the given category. The areas of the cerebral cortex relevant to the performing the task of this embodiment are approximately the same as those of the previous embodiment. The importance of each area in this embodiment may differ from that in the previous embodiment.

The classification of this embodiment was executed by using a data combination that is changed depending on the disorder. This is because the inventors considered the fact that, in the classification used in the previous embodiment, the measured data combinations that resulted in the highest coincidence ratios of the disorders with the diagnosis labels differed from one diagnosis to another. A classification was executed on a total of 121 test subjects (specifically, 55 NC subjects, 30 SC patients, 26 DP patients, and 10 BP patients) in the order of BP→SC→DP and NC. Which piece of data had to be used at each stage was determined on the basis of the coincidence ratios of various data combinations with diagnosis labels shown in Table 1. FRONT, LEFT, and RIGHT in Table 1 refer respectively to the frontal lobe, the left temporal lobe, and the right temporal lobe. In addition, the highest coincidence ratio for each group is shown in boldface. For BP, the data on the right temporal lobe were used at the first stage (a classification model shown in FIG. 5 with C1=0.32, C2=0.12, C3=−0.60, C4=−0.72, and thr_1=0.485 was used), and the data on the left temporal lobe were used both at the second stage and third stages (a model shown in FIG. 6 with thr_a=−0.0015 and thr_b=10 was used at the second stage; a model shown in FIG. 8 with D1=0.25, D2=0.35, D3=0.90, D4=0.07, and thr_1=0.117 was used at the third stage). For SC, the data on the frontal lobe were used at the first stage (a classification model shown in FIG. 5 with C1=0.22, C2=0.4, C3=−0.70, C4=−0.55, and thr_1=0.455 was used), and the data on the left temporal lobe were used both at the second and third stages (a model shown in FIG. 6 with thr_a=−0.0010 and thr_b=14 was used at the second stage; a model shown in FIG. 8 with D1=0.15, D2=0.25, D3=0.91, D4=0.29, and thr_1=0.107 was used at the third stage). For DP and NC, the data on the frontal lobe were used at the first stage (a classification model shown in FIG. 5 with C1=0.31, C2=0.12, C3=−0.59, C4=−0.74, thr_1=0.488 was used), and the data on the right temporal lobe were used both at the second and third stages (a model shown in FIG. 6 with thr_a=−0.0020 and thr_b=16 was used at the second stage; a model shown in FIG. 8 with D1=0.15, D2=0.45, D3=0.88, D4=0.02, thr_1=0.155 was used at the third stage). Thus obtained was a result of NC: 35/55 (64%), SC: 20/30 (67%), DP: 17/26 (65%), and BP: 6/10 (60%).

TABLE 1
Coincidence Ratio of Measured Data Combination with Diagnosis Label

| | |
|---|---|
| | Coincidence Ratio |
| Measured Area | with Diagnosis |
| for Employed Data | Label (%) |

| Stage 1 | Stage 2 | Stage 3 | NC | SC | DP | BP |
|---|---|---|---|---|---|---|
| FRONT | FRONT | FRONT | 64 | 50 | 42 | 40 |
| FRONT | FRONT | LEFT | 64 | 60 | 42 | 30 |
| FRONT | FRONT | RIGHT | 64 | 47 | 42 | 50 |
| FRONT | LEFT | FRONT | 64 | 63 | 62 | 50 |
| FRONT | LEFT | LEFT | 64 | 67 | 62 | 30 |
| FRONT | LEFT | RIGHT | 64 | 47 | 62 | 30 |
| FRONT | RIGHT | FRONT | 64 | 40 | 65 | 20 |
| FRONT | RIGHT | LEFT | 64 | 40 | 65 | 20 |
| FRONT | RIGHT | RIGHT | 64 | 57 | 65 | 40 |
| LEFT | FRONT | FRONT | 45 | 40 | 38 | 40 |
| LEFT | FRONT | LEFT | 45 | 37 | 38 | 30 |
| LEFT | FRONT | RIGHT | 45 | 30 | 38 | 30 |
| LEFT | LEFT | FRONT | 45 | 37 | 23 | 30 |
| LEFT | LEFT | LEFT | 45 | 30 | 23 | 20 |
| LEFT | LEFT | RIGHT | 45 | 30 | 23 | 30 |
| LEFT | RIGHT | FRONT | 45 | 23 | 23 | 20 |
| LEFT | RIGHT | LEFT | 45 | 47 | 23 | 30 |
| LEFT | RIGHT | RIGHT | 45 | 50 | 23 | 30 |
| RIGHT | FRONT | FRONT | 38 | 30 | 42 | 60 |
| RIGHT | FRONT | LEFT | 38 | 27 | 42 | 50 |
| RIGHT | FRONT | RIGHT | 38 | 23 | 42 | 40 |
| RIGHT | LEFT | FRONT | 38 | 23 | 31 | 30 |
| RIGHT | LEFT | LEFT | 38 | 30 | 31 | 60 |
| RIGHT | LEFT | RIGHT | 38 | 37 | 31 | 20 |
| RIGHT | RIGHT | FRONT | 38 | 30 | 27 | 30 |
| RIGHT | RIGHT | LEFT | 38 | 37 | 27 | 50 |
| RIGHT | RIGHT | RIGHT | 38 | 47 | 27 | 40 |

Lastly, an example of simple classifications of disorder will be described below. In clinical practice, there is a simple case where two different disorders have to be distinguished from each other. For example, there is a case where depression and bipolar disorder have to be distinguished from each other. By using the normalized parameters (integral (area), slope, and center of balance), the following calculation is performed:

Z=2*integral (area)+5*slope−2*center of balance

In this event, the values obtained by calculating by use of the data measured in the frontal lobe, the data measured in the left temporal lobe, the data measured in the right temporal lobe are expressed respectively by Z_front, Z_left, and Z_right. For depression patients, a relationship (Z_front+Z_left)/2<Z_right tends to hold true. For bipolar disorder patients, a relationship (Z_front+Z_left)/2>Z_right tends to hold true. By use of these relationships, 20 depression patients and 15 bipolar disorder patients were classified. Fifteen of the 20 depression patients were decided correctly whereas ten of the 15 bipolar disorder patients were decided correctly.

As has been described thus far, the use of the data measured in plural areas allows an effective classification of disorders to be executed.

### INDUSTRIAL APPLICABILITY

The present invention can be used for an apparatus for supporting and checking diagnosis of disorders such as psychiatric disorders.

1. An apparatus for measuring biological light characterized by comprising:
a part for biometry which measures hemoglobin changing waves by irradiating a plurality of areas of a head of a test subject with light beams having wavelengths from a visible range to an infrared range, and then by detecting the light beams having passed through an inside of the test subject;
a part for calculating characteristics which extracts a plurality of kinds of characteristic parameters from the hemoglobin changing waves measured respectively in the plurality of areas;
a part for decision which makes a decision of disorder by use of the plurality of kinds of characteristic parameters extracted by the part for calculating characteristics and in accordance with predetermined classification models; and
a display which displays the decision result obtained by the part for decision,
the apparatus characterized in that the part for decision includes, as the classification models, a plurality of classification models having a stratified structure.

2. The apparatus for measuring biological light according to claim 1, characterized in that at least one of the plurality of classification models uses a variable obtained by synthesizing the plurality of kinds of characteristic parameters.

3. The apparatus for measuring biological light according to claim 2, characterized in that the variable is a linear combination of the plurality of kinds of characteristic parameters.

4. The apparatus for measuring biological light according to claim 1, characterized in that the classification model of the highest stratum of the plurality of classification models having the stratified structure is a classification model for classifying a test subject into any of a normal control group and a non-normal control group.

5. The apparatus for measuring biological light according to claim 4, characterized in that, in the classification model of the highest stratum, a classification is executed by use of data measured in the frontal lobe.

6. The apparatus for measuring biological light according to claim 1, characterized in that
the classification model of the highest stratum of the plurality of classification models having the stratified structure is a classification model for classifying a test subject into any of a normal control group and a non-normal control group, and
a classification model at a lower stratum of the plurality of classification models is a classification model for ultimately classifying a test subject having been classified into the non-normal control group into one of a plurality of disorders.

7. The apparatus for measuring biological light according to claim 1, characterized in that the classification models are determined so as to maximize a probability that each of a plurality of test subjects having a definite decision of disorder is classified into a type corresponding to the decided disorder.

8. The apparatus for measuring biological light according to claim 1, characterized in that the characteristic parameters include any of a single characteristic parameter and a plurality of characteristic parameters selected from a slope of the hemoglobin temporal wave immediately after task start, an integral (area) during task including word recall, a second peak area after task, and a center of balance for the entire wave.

9. The apparatus for measuring biological light according to claim 1, characterized in that
the part for biometry has a multi-channel structure to measure a plurality of waves at a plurality of measurement positions of the test subject,
the part for calculating characteristics extracts characteristic parameters from each of the waves obtained respectively by the multiple channels, and
the maximum value of the extracted characteristic parameters is used as a target for analysis.

10. The apparatus for measuring biological light according to claim 1, characterized in that the part for decision includes a means for optimizing the classification models on the basis of a wave measured in a test subject having a definite diagnosis of disorder and/or a plurality of kinds of characteristic parameters extracted from the wave.

11. The apparatus for measuring biological light according to claim 1, characterized in that a combination of areas from which to extract the characteristic parameters used in the plurality of classification models having the stratified structure is changed depending on a disorder.

12. The apparatus for measuring biological light according to claim 1, characterized by comprising a means for specifying an area from which to extract the characteristic parameters.

13. The apparatus for measuring biological light according to claim 1, characterized in that each of the plurality of classification models having the stratified structure performs a classification by use of data measured in a corresponding specified area.

14. The apparatus for measuring biological light according to claim 2, characterized in that
the variable is a linear combination of a slope of the hemoglobin temporal wave immediately after task start, an integral (area) during task including word recall, a second peak area after task, and a center of balance for the entire wave.

15. The apparatus for measuring biological light according to claim 2, characterized in that
the variable is a linear combination of a slope of the hemoglobin temporal wave immediately after task start, an integral (area) during task including word recall, and a center of balance for the entire wave.
